(19) United States
(12) Patent Application Publication
(10) Pub. No.: US 2016/0206769 A1
(43) Pub. Date: Jul. 21, 2016

(54) ANTIMICROBIAL POLYMER WITH PARTICLES INCORPORATED THEREIN AND DISINFECTING ELEMENT BASED ON SAID POLYMER

(71) Applicant: TECNOLOGIES DEL CUORE CU, SL, Encamp (AD)

(72) Inventors: Marco ZEIGER, Elversberg (DE); Andreas SCHNEIDER, Saarbrücken (DE); Christian Amadeus FRONTZEK, Hanau (DE)

(21) Appl. No.: 14/898,328

(22) PCT Filed: Jun. 12, 2014
(86) PCT No.: PCT/EP2014/001592

§ 371 Date: Dec. 14, 2015

(30) Foreign Application Priority Data
Jun. 12, 2013 (EP) 13002999.4

(51) Int. Cl. A61L 2/232; A01N 59/20

(52) U.S. Cl. CPC A61L 2/232; A01N 59/20

(57) ABSTRACT

A disinfection element comprising a copper-based microbicidal contact region is intended to allow a particularly wide range of possible uses together with a reliably high level of microbicidal effectiveness in the contact region thereof. For this purpose, according to the invention, the contact region comprises a microbicidal surface layer formed of copper particles embedded in a matrix.

The invention relates to a disinfection element comprising a microbicidal contact region. The invention further relates to a method for producing a disinfection element of this kind.

In daily life, people frequently inadvertently come into contact with bacteria, germs and viruses which are harmful to health. In particular in places where people come into contact with surfaces which have previously been touched by a number of other people, an increasing accumulation of germs can lead increasingly to unhygienic conditions. In particular in public institutions and on public transport, people come into contact with objects such as door handles and other contact surfaces on which bacteria can collect and multiply. Pathogens can be transmitted and thus damage health even in institutions which actually follow strict hygiene regulations, such as in hospitals and kindergartens.

In order to counteract this, objects of this kind can be microbicidally equipped in the sense that surfaces or surface regions can be designed so as to be germicidal or bactericidal. For this purpose, a disinfection element comprising a corresponding microbicidal or germicidal contact region can be provided. A disinfection element of this kind is based on the design whereby, in the contact region provided for contact, for example the surface of a handle, a button or the like, the outer or exposed surface, and thus the surface which is subject to the contact, is provided with an appropriately selected microbicidal material, so that the germs or microorganisms which accumulate due to the repeated instances of contact are immediately killed again and thus right from the outset cannot become fully established.

A disinfection element of this kind, comprising a microbicidal contact region, is known for example from DE 10 2009 013 029 A1. The design of this known disinfection element is based on the use of copper as the base material for the desired microbicidal effect.

U.S. Pat. No. 7,445,799 discloses a composition for equipping woven fabrics, for example items of clothing, which composition is intended to provide a certain amount of protection against biological and chemical weapons. For this purpose, the woven fibres are provided with a surface coating in which inter alia metal microparticles are also incorporated.

The use of copper is also considered to be particularly advantageous in the present case, since copper especially has a number of properties which are particularly favourable for this use. Specifically, on the one hand, even in small amounts, copper as a material has a toxic effect for many microorganisms and can thus be used in a particularly reliable manner for killing germs or microorganisms. On the other hand, however, copper, in contrast with silver or other microbicidal basic materials for example, is a component of the natural human metabolism, and is therefore in principle harmless to the human organism.

There is therefore no need to fear that the human organism may be contaminated by a copper-based disinfection element, even in the case of a strong microbicidal effect. The following embodiments therefore relate to the use of copper as the microbicidal base material, which is considered to be particularly advantageous. In cases in which particularly good compatibility with the human organism is not a high priority, in place of copper another suitable microbicidal basic material such as silver, zinc or the like, or a mixture of said materials, can in principle also be provided in a similar embodiment which is likewise considered to be within the meaning of the present invention.

The above-mentioned properties of copper as a microbicidal base material are used in a targeted manner in the disinfection element according to DE 10 2009 013 029 A1. In this known disinfection element it is also taken into account in a targeted manner that, in order to achieve a sufficiently great microbicidal or germicidal effect, a minimum number of copper ions emitted (also referred to in the following as “ion release”) per unit of area and per unit of time to the woven surfaces which come into contact with the contact region should be maintained.

With this in mind, the disinfection element known from DE 10 2009 013 029 A1 is designed to release a particularly large number of ions in the contact region, and for this purpose has a copper-based surface structure in the contact region which has an inner surface which is larger than the outer surface. However, this design of the known disinfection element requires a comparatively very large construction of the exposed surface of the contact region. The known disinfection element therefore has only a limited amount of flexibility in terms of the possible fields of use thereof.

WO 2006/099906 A1 discloses a transparent, porous sol-gel layer which is doped with at least one antimicrobially active substance in the form of nanoparticles.

US 2007/0237946 A1 discloses a substrate which has an electron-emitting surface. In this case, metal particles are present in the surface, which consist of palladium and at least one metal selected from the group comprising gold, ruthenium, rhodium, osmium, iridium and platinum. The quantity of metal particles is between 0.001 and 0.8 μg/cm² and is conducive for use when preventing nosocomial infections.

WO 2012/135294 A2 discloses a coated glass, suitable in particular for touchscreen devices, which has an antimicrobial surface. The antimicrobial surface consists of Cu nanoparticles.

WO 2007/087792 A2 discloses an oligodynamically active device comprising a surface containing copper or silver and a moisture-permeable precious metal coating applied thereto. Said coating comprises a precious metal as the surface, in particular gold, palladium, rhodium, platinum or ruthenium, and has a thickness in the nanometre or micrometre range. Said coating is designed such that the surface containing copper or silver is in moisture contact with the precious metal and can gradually dissolve by means local element formation.

In each case, the known prior art is limited in terms of the manner of use thereof and the materials used. A flexible use on different surfaces is not guaranteed. A strong disinfecting effect is not necessarily guaranteed either.

The object of the invention is that of specifying a disinfection element of the type mentioned at the outset, which allows a particularly wide range of possible uses together with a reliably strong microbicidal effect in the contact region thereof. Furthermore, a method for producing a disinfection element of this kind is intended to be specified.

With regard to the disinfection element, this object is achieved according to the invention in that the contact region has a microbicidal surface layer, formed of copper particles embedded in a matrix, wherein the copper particles in the surface region of the layer are at least in part not covered by the matrix material, and wherein the fraction of the overall surface of the surface layer formed by the exposed copper particles is at least 10%.

The invention is based on the consideration that, in view of the above-mentioned particularly favourable properties of copper for the intended use, copper should be provided unmodified as the base material for microbicidally equipping the contact region.

The sufficiently high ion release or ion emission, which is provided depending on the design and is also desirable, could be guaranteed firstly in that a material is advantageously selected for the matrix material which has a high degree of intrinsic permeability specifically for copper ions, and thus if possible only slightly impedes ion migration of copper ions. Alternatively or in addition, the ion emission is also promoted in that the contact region of the disinfection element is designed in such a way that the copper particles in the surface region of the coating are at least in part not covered by the material matrix so that, if required, direct mechanical contact is possible between the particles and germs which are becoming established.

As has been found, in fact completely surprisingly, in addition to the ion emission, the direct contact of the germs with copper or the copper particles is also particularly favourable for the antimicrobial activity. Specifically due to the above-mentioned particle sizes, a compromise, which is considered to be particularly favourable, between high ion emission and as large as possible an exposed contact face on the surface of the disinfection element can be achieved. It is presumed that open or exposed copper faces on the contact surface result in damage to the cell envelope of the microbes and thus promote the penetration of the copper ions into the inside of the cell. Consequently, a different material can also be selected as the contact material for the microbes, which material likewise results in such a targeted weakening of the cell envelope.

Preparing uncovered or exposed copper faces of this kind can be achieved when the dimensioning parameters (in particular particle size in combination with the thickness of the coating) are appropriately selected, in that the copper particles protrude upwards and uncovered out of the matrix material at least in part.

A process of roughening and/or cauterising the matrix, by means of which the matrix material above the incorporated copper particles is removed at least in part, can also thus expose the surface of said particles at least in part. This is carried out by means of a mechanical process, for example grinding. The microbicidal effect of a material compound produced in this way, having a exposed copper fraction on the surface, in particular has the advantage that, in this respect, the ion emission or ion release, or the direct contact between the cell wall and the copper face are not impeded by the material matrix.

For the desired sufficiently high ion release and/or the exposed contact region, it is provided for the fraction of the overall surface of the coating formed by the exposed copper particles to be at least 10%.

Advantageously, when designing the disinfection element, in addition to the effectiveness thereof (which can be achieved by sufficiently high ion release or emission and/or by contact which is, if possible and to a large extent, direct and mechanical between the partially embedded copper and the germs becoming established), the mechanical resilience and thus the long-time stability thereof even in the presence of abrasion or other mechanical stresses is in particular taken into account. Within the meaning of sufficiently high mechanical stability, the matrix material, which produces the mechanical stability among the copper particles in the manner of a binder, should occupy an appropriately selected volume fraction of the coating as a whole. In order to take account thereof, the volume fraction of the copper particles is advantageously of between 10% and 80% of the overall volume of the coating.

However, in order to allow a particularly high degree of flexibility in use and in the configuration of the contact region, the use of a very large copper block should be avoided.

Instead, it is provided to incorporate the copper, provided in order to make available the microbicidal properties, in a matrix made of an appropriately selected substrate. Said substrate should be present in particular in a form which is initially fluid but can later be hardened, with the result that the matrix material provided with copper particles can be shaped in a particularly simple manner. In this way, it is possible to reliably equip even relatively complex geometries with a microbicidal surface layer and thus a particularly large variety of possible spatial configurations of the disinfection element or of the contact region thereof is possible.

In this case, the disinfection element can be formed in the manner of a solid body, as a filled body which is formed entirely of the hardened matrix material provided with the embedded copper particles.

In this case, the disinfection element can be produced for example in the manner of a cast body, it being possible during production to draw on conventional methods appropriately selected for the matrix material provided in each case. In the process, the disinfection element formed in this way can be provided with an at least approximately spatially homogeneous, uniform distribution of the embedded copper particles, in which all the spatial regions of the disinfection element comprise a comparable concentration of embedded copper particles. However, in a particularly advantageous embodiment, the concentration of the embedded copper particles can also be spatially inhomogeneous and superelevated in the region close to the surface, so that most of the fraction of the copper particles is positioned close to the surface of the contact region.

A concentration curve of the copper particles of this kind, which is selected in a targeted manner, can be obtained for example in that the copper particles are mixed into a starting material provided for forming the matrix and are temporarily held therein in suspension. During hardening of the starting material in order to form the matrix, the copper particles can sink to the bottom on account of gravity, resulting in an accumulation of the particles in the lower interface region of the developing cast body. Said lower interface region can then be used for forming the contact region.

In this case, the distribution of the particles in the case body can be set in a manner particularly suited to the requirements by appropriately selecting the parameters during production. A desired concentration of the particles in the region close to the surface can be set in a reliable and reproducible manner in particular by appropriately selecting the process times, i.e. in particular the time selected for concentrating the particles in the interface region, in view of the material properties of the starting material provided for forming the matrix, in particular the viscosity thereof.

In an alternative advantageous embodiment, the microbicidal surface layer of the contact region is designed as a coating applied to a substrate. A particularly high degree of flexibility, in particular in terms of possible geometric shapes, can thereby be achieved. In this case, incorporating the copper particles in the matrix material allows a particularly flexible use of the microbicidal material even on differently shaped or designed surface regions of the substrate. When embedding the copper particles into the matrix material, advantageously care is taken in particular to ensure that there is sufficiently high ion release of copper ions towards the exposed outer surface of the contact region, so that the microbicidal effect can be reliably maintained. This is achieved on the one hand by the above-mentioned concentration of copper particles close to the surface. On the other hand, however, as has surprisingly been found, this can also be achieved in a particularly favourable manner in that, in an advantageous embodiment, the copper particles are present in the matrix as what are known as nanoparticles or microparticles, i.e. as particles having an average size in the nanometre or micrometre range.

In a particularly advantageous embodiment, the copper particles embedded in the matrix thus comprise a particle fraction having a particle size of at most 500 nm, particularly preferably having a particle size of from 90 to 250 nm, and/or a particle fraction having a particle size of at least 1 μm. In this case, the particle fraction particularly preferably has an average particle size of from approximately 20 μm to 50 μm and/or a standard deviation of at most 20%. Such a selection of the particle size in the nanometre or micrometre range ensures inter alia that the particles have a particularly high surface-volume ratio and thus a particularly large specific surface area, which greatly promotes the ion emission.

With regard to a particularly favourable processability and applicability of the copper-containing microbicide provided, the material provided for forming the matrix is expediently selected in a targeted manner. In this case, on the one hand account should be taken inter alia of the ability to reliably and, if required, also uniformly disperse the copper particles, so that if possible no, or just a little, agglomeration occurs.

On the other hand, however, particularly good processability during shaping, i.e. when producing a cast body and/or when applying the coating, should also be aimed for. For this purpose, in particular the presence of a starting material as a liquid in which the copper particles can be introduced and which can then be hardened following the shaping or coating, is desirable.

In view of these criteria, the matrix is advantageously formed of a polymer material, preferably of organic or inorganic structural elements, (poly)para-phenylenes, an epoxy resin and/or polyurethane.

The surface layer forming the contact region of the disinfection element expediently has a layer thickness of at least 1 μm, in particular of at least 10 μm, and of at most 100 μm. These dimensions are particularly favourable since, on the one hand, a certain minimum number of copper particles is required for killing the microorganisms or germs.

On the other hand, however, precisely in view of the intended particle size of the copper microparticles, the thickness should not be selected to be too great, so that at least part of the particle surface can, in a particularly simple manner, be kept uncovered by the matrix material.

With regard to the production method for the disinfection element, the above-mentioned object is achieved in that copper particles are stirred into a solution of a preferably polymer-based matrixing agent, and the suspension or dispersion thereby obtained is subsequently homogenised. This starting material can then be subjected to shaping and subsequently hardened so that a cast body provided for forming the disinfection element or the contact region thereof develops. Alternatively, a substrate can be coated at least in part with a coating solution provided with the copper particles and the matrixing agent. In a particularly advantageous embodiment, a coating process by precipitating a coating solution, provided with copper particles and a matrixing agent, onto a substrate by means of dip coating to form the contact region is provided for this production of the disinfection element.

In order to reliably ensure the effectiveness of the produced coating, and in particular to make possible a sufficiently high ion release, optionally together with a large contact face, the copper particles are advantageously stabilised before being introduced into the solution of the matrixing agent, with the result that agglomeration of the particles is largely prevented and a particularly large outer surface of the copper particle fraction is produced. In the process, the particles are preferably dispersed in a fluid, preferably ethanol, by means of ultrasound treatment, so as to be largely present in the form of separated individual particles.

Subsequently, the surface of the copper particles is functionalised by an appropriately selected chemical component, preferably polyvinylpyrrolidone (PVP), so that clogging or agglomeration is prevented (what is known as steric stabilisation). In order to achieve a thin layer on the substrate, in an advantageous variant said layer can be applied for example by means of dispersion coating, the material used in the coating method being finely distributed in a solvent in the form of a dispersion.

The dispersion is atomised to a mist by means of compressed air, and is uniformly sprayed onto the substrate. The substrate is subsequently heated in an oven so that a thin layer is formed.

The advantages achieved by the invention consist in particular in that, by equipping the matrix material, in particular a polymer, with embedded copper particles, a material can be provided which can be processed comparatively well and flexibly, which material has an antimicrobial or microbicidal effect and can thus be used particularly favourably and reliably for disinfection purposes. In the process, the particularly large amount of flexibility especially during final processing by means of appropriate shaping or by application in the form of a coating, allows a large variety of conceivable use possibilities and fields of use.

By embedding the copper particles, in particular copper nanoparticles or microparticles, in a matrix material, high copper ion release can be ensured, even for contact regions having relatively complex geometries or in large quantities. This makes it possible to reliably provide a microbicidal contact region in a particularly simple manner, even in the case of flexible or varying requirements.

An embodiment of the invention will be described in greater detail on the basis of figures, in which:

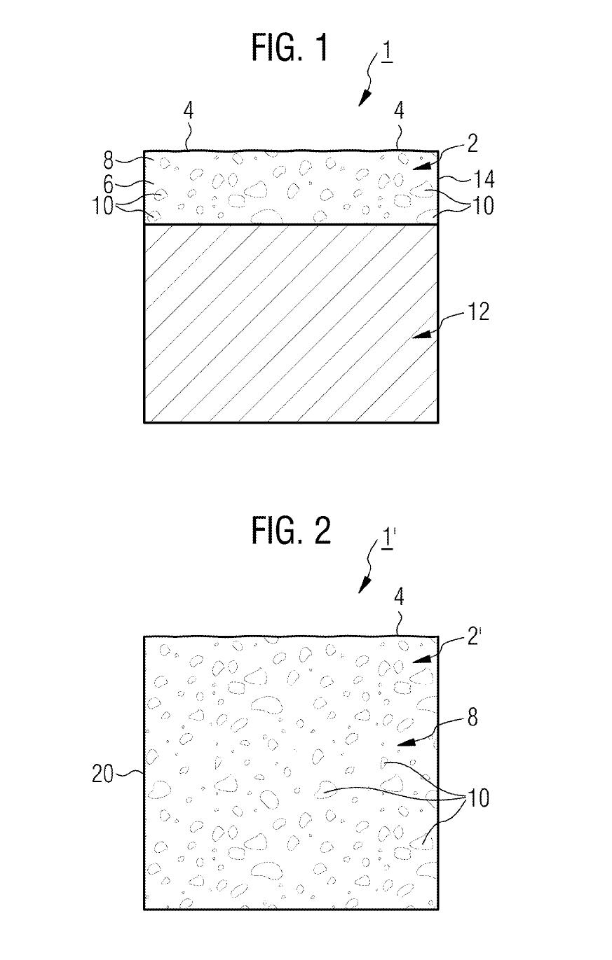

FIG. 1-4 are each cross sections of a disinfection element,

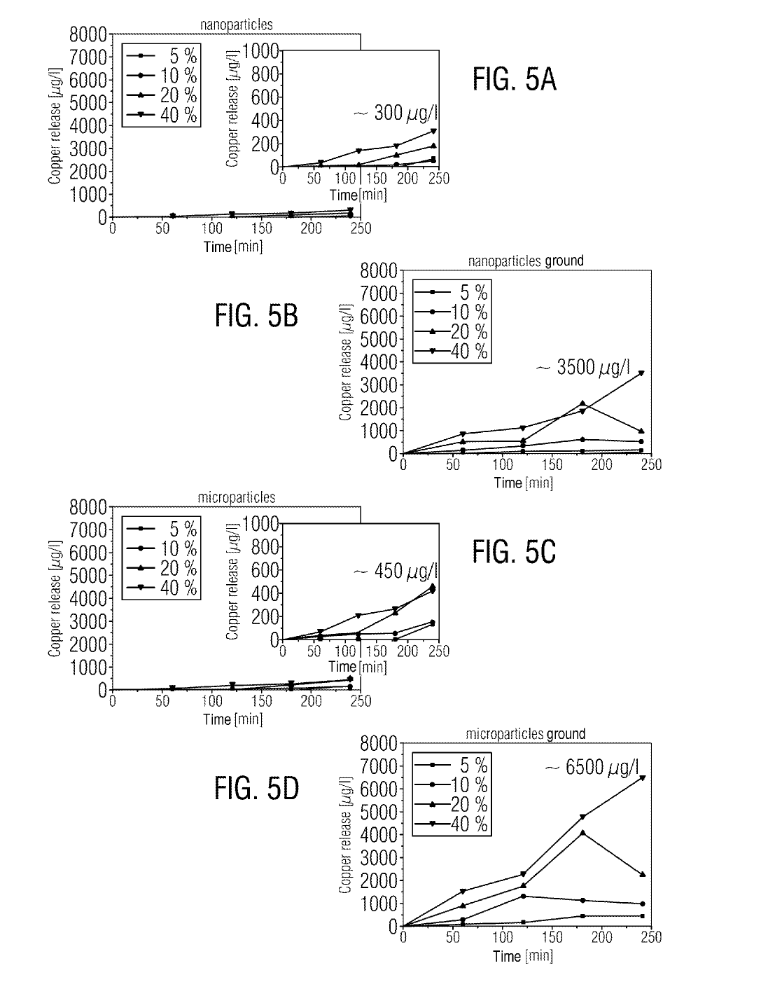

FIG. 5-7 are each graphs showing the progression over time of the copper ion release, and

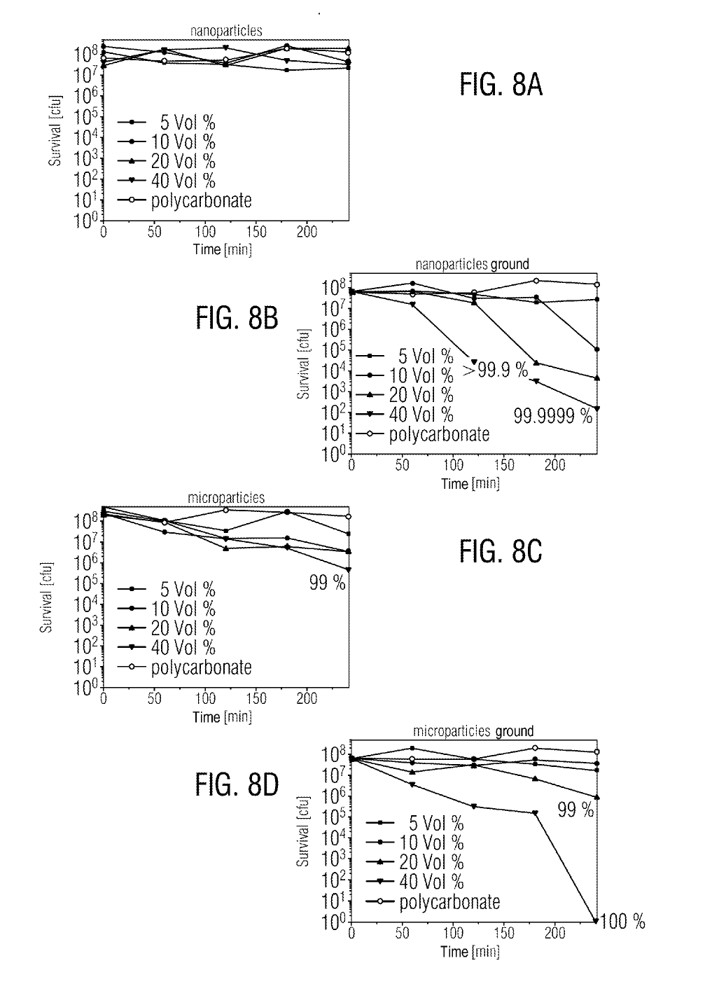

FIG. 8-10 are each graphs showing the progression over time of the number of bacteria.

Identical parts are provided with the same reference signs in all the figures.

The disinfection element according to FIG. 1 is provided for use on or in objects or surfaces in general, where there are likely to be frequent instances of contact by a large number of people, such as door handles or handrails in public buildings, or where a particularly high level of asepsis or sterility is to be ensured for other reasons, such as in hospitals.

In order to combat germ development or contamination by microorganisms in a targeted and consistent manner, the disinfection element 1 is designed to be microbicidal or germicidal in the contact region 2 thereof provided to be touched by people. In this case, the surface region of the contact region 2 is designed using copper as a microbicidal base material, in order to be able to make targeted use of the properties of copper, which are particularly favourable for this purpose. In this case, targeted use should be made in particular of the fact that, on the one hand, even in small amounts copper has a toxic effect for many microorganisms, meaning that it can be used in a particularly reliable manner for killing germs or microorganisms, but on the other hand copper, in contrast with silver or other microbicidal basic materials for example, is a component of the natural human metabolism, and is therefore in principle harmless to the human organism.

The disinfection element 1 is designed to have a particularly high degree of flexibility and variable use possibilities. In order to make this possible, the contact region 2 has a microbicidal surface layer 6 on the surface 4 thereof which is freely accessible and therefore contactable.

Said microbicidal surface layer 6 is formed by a matrix 8 in which copper particles 10 are embedded as the actual microbicidal material.

In view of the desired operation, i.e. the targeted use of the microbicidal properties of the copper embedded in the matrix 8, the surface layer 6 is specifically designed for a sufficiently high release of copper ions to the surface region. As has surprisingly been found, this can be achieved in a particularly favourable manner in that the copper particles 10 are present in the matrix 8 as what are known as nanoparticles or microparticles, i.e. as particles having an average size in the nanometre or micrometre range respectively.

Accordingly, in the embodiment, the copper particles 10 embedded in the matrix 8 have an average particle size of approximately 20-50 μm, having a standard deviation of at most 20%. In an alternative advantageous embodiment, the copper particles 10 can also comprise a particle fraction having a particle size of at most 500 nm, preferably having a particle size of from 90 to 250 nm.

The copper particles 10 can be designed to be largely homogenous among themselves and as filled bodies, and can consist in particular of pure or virtually pure copper, and optionally also of an appropriately selected alloy. Alternatively and preferably, however, a particularly material-saving use of the copper is conceivable, in that the copper particles 10 are in turn designed as particles of a substrate which are coated on the outside with copper. Said particles can be coated on the outside with a copper envelope, for example by drawing on the fluidised bed technique.

The fluidised bed technique makes it possible, in a similar manner to plasma coating but in particular for very small particles, to coat on an industrial scale. For the specific use, i.e. in particular coating with copper, in particular expanded clay or also similarly lightweight metals and/or metals which themselves have an antimicrobial effect are particularly suitable and therefore particularly preferred as the base material or substrate. In addition to a particularly effective use of resources and the cost reduction which can thereby be achieved, an improved surface quality in the finished produce and/or an optimisation of the operating mechanism can also be achieved by such a design of the particles 10.

With regard to the design of the surface layer 6, a plurality of variants are conceivable, which are considered to be particularly advantageous. In the embodiment according to FIG. 1, the surface layer 6 is designed as a coating 14 applied to a substrate 12.

The material provided for forming the matrix 8 is selected in a targeted manner in view of favourable processability and applicability. Specifically, in particular for producing the contact region 2 or the surface layer 6 thereof, it is provided to apply the matrix 8 containing the copper particles 10 by means of a dip coating method.

Accordingly, the starting material provided for forming the matrix 8 is selected in a targeted manner taking account of the design parameters, such that, inter alia, on the one hand the copper particles 10 are reliably and optionally also uniformly received, and that on the other hand particularly good processability for applying the coating 14 is ensured. In this case it is provided that, following appropriate preparation, in particular following a stabilisation step, the copper particles 10 are introduced into a liquid and homogenised and uniformly distributed therein. Subsequently, the fluid loaded with the particles 10 is intended to be applied to the substrate 12 in the context of dip coating, and subsequently a hardening step, optionally assisted by heat treatment, is provided for forming the actual matrix 8. In view of these criteria, the starting material provided for forming the matrix 8 is a polymer material, preferably selected from organic or inorganic structural elements, para-phenylenes, an epoxy resin and/or polyurethane.

In the alternative embodiment according to FIG. 2, the disinfection element 1′ is, in contract, designed, at least in the contact region 2′ thereof, as a cast or filled body 20.

Said body is formed by the hardened matrix material provided with the embedded copper particles 10. Also in view of these criteria, the starting material provided for forming the matrix 8 is likewise a polymer material, preferably selected from organic or inorganic structural elements, para-phenylenes, an epoxy resin and/or polyurethane. Said materials are thus equally suitable and preferred as matrixing agents for both variants, i.e. for producing a coating 14 and for producing a cast or filled body. When producing the filled body 20, it is possible to draw on conventional methods which are appropriately adapted to the matrix material used.

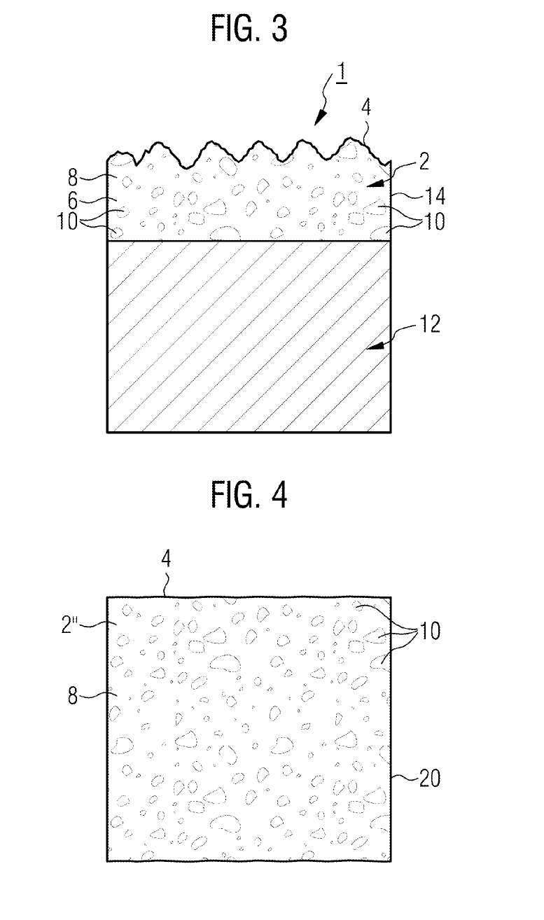

In the embodiments shown in FIGS. 1 and 2, the copper particles 10 are essentially completely embedded in the matrix 8 and enclosed thereby. However, in order to still further promote the sufficiently high ion release or ion emission, which is provided depending on the design and is also desirable, the contact region 2 of the disinfection element 1 can be designed in a particularly preferred embodiment, shown in FIG. 3, such that the copper particles 10 in the surface region of the surface layer 6 are at least in part not covered by the matrix 8. In addition to an ion release in the exposed region of the particles which is increased overall, this allows direct contact between (germ) cell envelopes and copper atoms, with the result that the cell envelopes can be weakened or damaged in a targeted manner. In order to produce the corresponding surface regions, a process of roughening and/or cauterising the matrix 8 is preferably provided, by means of which the matrix material above the incorporated copper particles 10 is removed at least in part and the surface of said particles is thus exposed at least in part. Roughening the surface by means of mechanical grinding has proven particularly reliable and thus particularly preferred for this purpose.

In the embodiment, and particularly preferably, the fraction of the overall surface of the surface layer 6 which is formed by the exposed copper particles 10 is at least 10%.

In the embodiments according to FIGS. 1 to 3, the surface layer 6 is designed having an at least approximately spatially homogeneous, uniform distribution of the embedded copper particles 10, in which all the spatial regions of the surface layer 6 comprise a comparable concentration of embedded copper particles 10.

However, in the alternative embodiment according to FIG. 4, which is also considered to be particularly advantageous, the concentration of the embedded copper particles 10 is spatially inhomogeneous, a superelevation being present in the region close to the surface 4, so that most of the fraction of the copper particles 10 is positioned close to the surface 4 of the contact region 2″. A concentration curve of the copper particles 10 of this kind, which is selected in a targeted manner, can be obtained for example in that the copper particles 10 are mixed into the starting material provided for forming the matrix 8 and are temporarily held therein in suspension. During hardening of the starting material in order to form the matrix 8, the copper particles 10 can sink to the bottom on account of gravity, resulting in an accumulation of the particles in the lower interface region of the developing cast body. Said lower interface region can then be used for forming the contact region 2″.

Within the meaning of sufficiently high mechanical stability, in all of the above embodiments an appropriately selected volume fraction of the overall surface layer 6 is provided for the matrix material, which produces the mechanical stability among the copper particles 10 in the manner of a binder. In order to take account thereof, the volume fraction of the copper particles 10 in the embodiment is of between 5% and 40% of the overall volume of the surface layer 6.

The production of the disinfection element 1 is more or less identical for all variants, apart from the provision of the freely accessible surface of the copper particles 10, and apart from in the case where a filled body 20 or a coating 14 is desired. Therefore, in the following, some examples of the production of the coating 14 will be explained in further detail, which examples can also be transferred without difficulty to the production of filled bodies 20 in terms of the results aimed for, optionally by drawing on appropriate standardised casting methods. In particular, for producing the coating 14 using a dip coating method, a process corresponding to the examples described below has proven particularly advantageous.

1. Preparation of the Substrate 12:

As a trial, a steel substrate (low carbon steel), a polyethylene substrate and a glass substrate were coated as the substrate 12.

Prior to coating, roughening was carried out in order to improve the adherence of the coating 14. Subsequently, the substrates were cleansed using isopropanol and distilled water and dried for a period of 24 hours.

2. Stabilisation of the Copper Particles 10:

For the purpose of stabilisation, the copper particles 10 were first subjected to a treatment step by means of ultrasound in ethanol for 4 minutes. Subsequently, polyvinylpyrrolidone (PVP) was added to the mixture and ultrasound treatment was again carried out for a period of approximately 4 minutes. Subsequently, drying was carried out at a temperature of 90° C. for a period of 24 hours, the solvent being completely evaporated. The surface of the copper particles 10 is functionalised by means of this treatment with polyvinylpyrrolidone (PVP), what is known as steric stabilisation, with the result that clogging or agglomeration is subsequently prevented.

3. Mixing of the Matrixing Agent and Application of the Coating 14:

The stabilised copper particles 10 were mixed with the matrixing agent and subsequently applied to the substrate 12 by means of dip coating. The following alternatives were tested and identified as advantageous:

Matrixing Agent Poly Para-Phenylenes (PPP):

The matrixing agent PPP (available for example as “PrimoSpire PR-250” from the manufacturer Solvay Specialty Polymers) was dissolved in the required amount in chloroform. After mixing, the mixture was initially stirred at a moderate speed (200 rpm) for 60 minutes in order to ensure complete dissolution. Subsequently, the required quantity of stabilised copper particles 10 was mixed in. Said mixture was stirred at high speed (6,500 rpm to 9,500 rpm) for a period of few minutes for the purpose of homogenisation. Subsequently, the substrate 12 was dipped in the homogenised solution for the purpose of dip coating, and then tried for 24 hours. In order to evaporate chloroform residues which may remain, heat treatment was then carried out at 90° C. in a vacuum for approximately 48 hours.

Matrixing Agent Epoxy Resin:

The required quantity of stabilised copper particles 10 was mixed into the matrixing agent (available as “EPON 28” for example). For the purpose of homogenisation, said mixture was stirred at high speed (6,500 rpm to 9,500 rpm) for a period of a few minutes and then left to cool. The required quantity of curing agent (for example EPIKUR 3223) was mixed in according to a resin/curing agent ration of 10:1 and the mixture was stirred by hand. Subsequently, the substrate 12 was dipped in the homogenised solution for the purpose of dip coating and subsequently dried for 24 hours. The resin formation was completed by subsequent heat treatment at 100° C. for approximately 4 hours.

Matrixing Agent Polyurethane (PU):

The matrixing agent PU (available for example as TECOFLEX RESIN EG65D from the manufacturer Lubrizol) was dissolved in the required quantity of solvent (for example n,n-dimethylformamide, DMF, available as ANHYDROUS 99.8% from the manufacturer Sigma Aldrich) and subjected to ultrasound treatment for 3 hours in order to ensure complete dissolution of the matrixing agent in the solvent. The required quantity of stabilised copper particles 10 was subsequently mixed in. For the purpose of homogenisation, said mixture was stirred at high speed (6,500 rpm to 9,500 rpm) for a period of a few minutes. Subsequently, the substrate 12 was dipped in the homogenised solution for the purpose of dip coating and subsequently dried for 24 hours. In order to evaporate solvent residues which may remain, heat treatment was then carried out at 60° C. in a vacuum for approximately 48 hours.

4. Results:

The antibacterial effect was tested on E. coli K12 (ATCC 23716) using a “wet plating” method [Wilks et al., 2005, Int. J. Food Microbiol. 105:445-454]. For this purpose, the bacteria culture was grown for 24 hours at 37° C. in Luria-Bertani medium to an optical density of between 2.1 and 2.3. 10 ml of this bacteria suspension having a concentration of approximately 109 per ml was centrifuged at 4000 rpm for 10 minutes. Finally, the centrifuged bacteria were then mixed by vortexing with 10 ml of 0.9% NaCl solution and diluted 1:10.

In order to test the antibacterial properties, 25 μl of the corresponding bacteria suspension having a specified concentration of approximately 10⁸ bacteria was added to the sample and removed again from the sample, using a pipette, after specified contact times of 60 minutes, 120 minutes, 180 minutes and 240 minutes. The respective dilutions of the removed samples were plated on LB agar plates and the colony-forming units were counted after 24 hours. A polycarbonate plate, which does not have any microbicidal properties, was used as a reference for all samples.

The copper ion emission was measured by means of atom absorption spectrometry on the same samples, in order to measure the emitted copper concentration in the bacteria suspensions, as well as the antibacterial effectiveness. Said concentration can be used, in some circumstances, for quantifying antibacterial properties of copper-based systems [Molteni et al., 2010, Appl. Environ. Microbiol., vol. 76 no. 12 4099-4101]. The results of the measurements for the copper ion emission are shown in FIG. 5-7, and the results for the microbicidal effect are shown in FIG. 8-10.

The graphs according to FIG. 5 show the copper emission (or “ion release”) as a function of time at different concentrations of unground copper nanoparticles (FIG. 5a), ground copper nanoparticles (FIG. 5b), unground copper microparticles (FIG. 5c) and ground copper microparticles (FIG. 5d). In this case, “unground” and “ground” indicate whether or not the coating 14, formed by the matrix 8 in which the copper particles 10 are embedded, was subjected to surface treatment (roughening) by means of mechanical grinding after hardening of the matrixing agent. In this case, the particles are embedded in poly para-phenylenes. The measurement was carried out by means of atom absorption spectrometry.

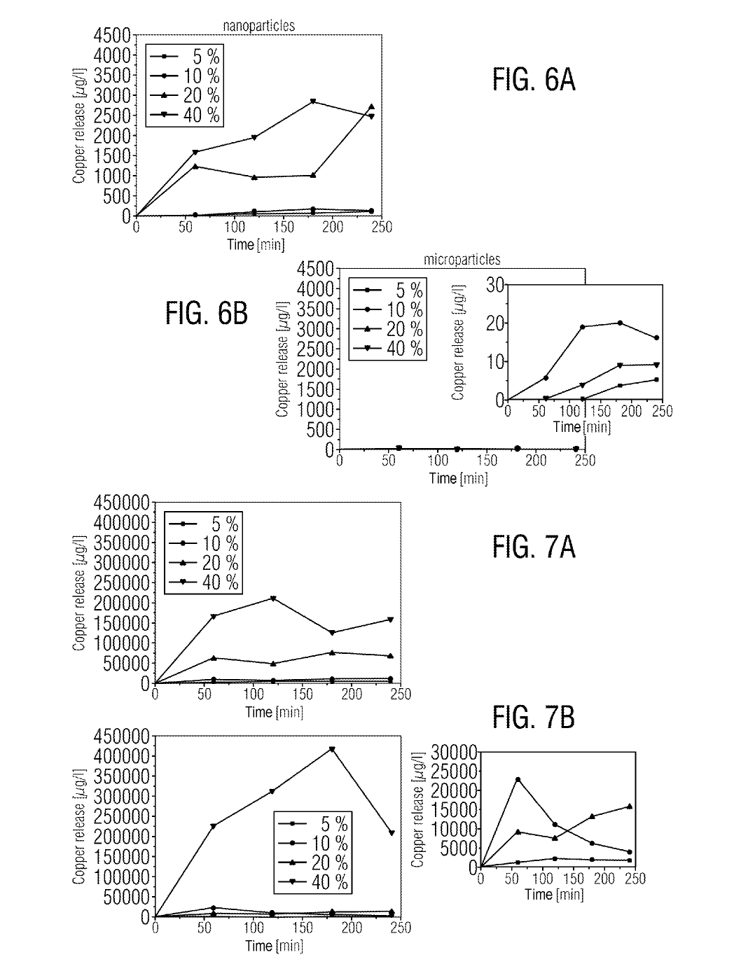

As a comparison, the graphs according to FIG. 6 show the copper emission as a function of time at different concentrations of copper nanoparticles (FIG. 6a) and copper microparticles (FIG. b) embedded in polyurethane. The measurement was carried out by means of atom absorption spectrometry.

In the graphs in FIG. 7, on the other hand, the copper emission as a function of time at different concentrations of copper nanoparticles (FIG. 7a) and copper microparticles (FIG. 7b) embedded in epoxy resin is shown. The measurement was carried out by means of atom absorption spectrometry.

The results of the measurements for the copper ion emission of different polymer-copper composites all show an increase in the copper concentration as a function of time, with the exception of the epoxy resin systems which reach their saturation point after 120 minutes. Furthermore, it can be seen that the copper ion emission also increases as the concentration of copper nanoparticles and copper microparticles increases. In this case, significant values cannot be identified at 5 vol. %.

In the case of poly para-phenylenes, the measurements show that microparticles (approximately 400 μg/l after 240 minutes at 40 vol. %) bring about similar or slightly higher ion emission compared with nanoparticles (approximately 300 μg/l after 240 minutes at 40 vol. %). Furthermore, a great increase in the copper concentration can be observed when the two samples are ground after production. This effect is quantified in an increase of more than one order of magnitude.

However, no significant effect can be identified for polyurethane in combination with microparticles (less than 20 μg/l for all concentrations), the nanoparticle composite bringing about a similar level of ion emission as the ground variant of poly para-phenylenes.

The ion emission in the case of epoxy resin is approximately two orders of magnitude higher compared with all other systems. Here, the effect in the case of microparticles (approximately 400 μg/l after 180 minutes at 40 vol. %) is greater by a factor of 2 than for nanoparticles.

It can therefore be noted that the polymer systems of poly para-phenylenes and epoxy resin have higher ion emission together with microparticles than said systems together with nanoparticles, and that pre-treatment of poly para-phenylenes by mechanical grinding can increase the copper ion emission many times.

In order to illustrate the microbicidal properties of the produced composite, the graph according to FIG. 8 shows the number of colony-forming units (surviving bacteria; in this case E. coli) as a function of time at different concentrations of unground copper nanoparticles (FIG. 8a), ground copper nanoparticles (FIG. 8b), unground copper microparticles (FIG. 8c) and ground copper microparticles (FIG. 8d), the particles having been embedded in poly para-phenylenes. The measurement was carried out by means of the “wet plating” method.

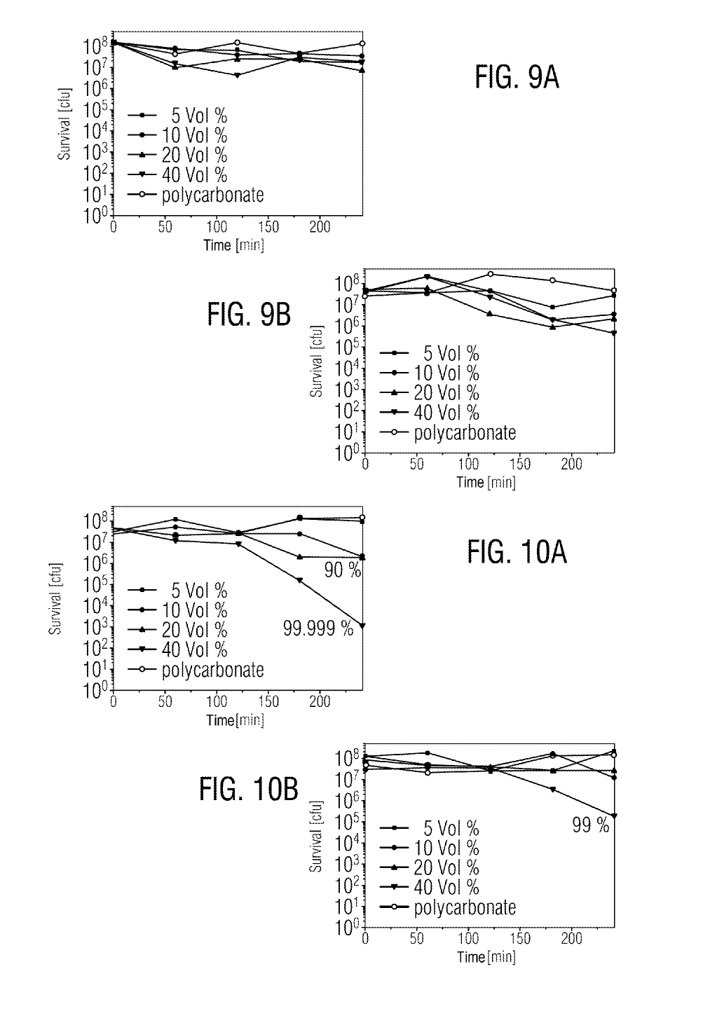

The graphs in FIG. 9 show the number of colony-forming units (surviving bacteria; in this case E. coli) as a function of time at different concentrations of copper nanoparticles (FIG. 9a) and copper microparticles (FIG. 9b), the particles having been embedded in polyurethane. This measurement was also carried out by means of the “wet plating” method. The graphs according to FIG. 10 in addition show the number of colony-forming units (surviving bacteria; in this case E. coli) as a function of time at different concentrations of copper nanoparticles (FIG. 10a) and copper microparticles (FIG. 10b), the particles having been embedded in epoxy resin. This measurement was also carried out by means of the “wet plating” method.

The results for the antibacterial effect against E. coli show that poly para-phenylenes and polyurethane as the matrixing agent do not exhibit any significant microbicidal effect either together with nanoparticles or together with microparticles. The ground poly para-phenylene samples, however, exhibit a significant microbicidal effect, which manifests itself in complete killing at 40 vol. % of microparticles after 240 minutes. Over 100 bacteria can be detected after 240 minutes when using nanoparticles at a concentration of 40 vol. %.

In comparison with poly para-pheynlenes, the epoxy resin composites also exhibit a significant effect without mechanical pre-treatment. The graphs in FIG. 10 show that only approximately 1000 bacteria were still detected at 40 vol. % when using nanoparticles. The use of microparticles together with otherwise identical parameters merely causes a reduction in the bacteria of more than two log steps.

Accordingly, it is possible to conclude that mechanical grinding of the polymer surface (using the example of poly para-phenylenes) can vastly increase the antibacterial effect, and that using microparticles in this case has a significantly greater effect than using nanoparticles. In addition, it could be shown that epoxy resins have a strong antibacterial effect, even without mechanical further processing, nanoparticles having a slightly quicker effect than microparticles.

### LIST OF REFERENCE NUMERALS

1, 1′ disinfection element

2, 2′ contact region

4 surface

6 surface layer

8 matrix

10 copper particles

12 substrate

14 coating

20 filled body

1. Disinfection element comprising a contact region which has a microbicidal surface layer, formed of copper particles embedded in a matrix, wherein the copper particles in the surface region of the layer are at least in part not covered by the matrix material, and wherein the fraction of the overall surface of the surface layer formed by the exposed copper particles is at least 10%.

2. Disinfection element according to claim 1, wherein the volume fraction of the copper particles in the overall volume of the surface layer is of between 10% and 80%.

3. Disinfection element according to claim 1, wherein the microbicidal surface layer is designed as a coating applied to a substrate.

4. Disinfection element according to claim 1, wherein the copper particles embedded in the matrix comprise a particle fraction having a particle size of at most 500 nm, preferably having a particle size of from 90 nm to 250 nm.

5. Disinfection element according to claim 1, wherein the copper particles embedded in the matrix comprise a particle fraction having a particle size of at least 1 μm.

6. Disinfection element according to claim 5, wherein the particle fraction has an average particle size of approximately 50 μm having a standard deviation of at most 20%.

7. Disinfection element according to claim 1, wherein the matrix is formed of a polymer material, preferably paraphenylenes, an epoxy resin and/or polyurethane.

8. Disinfection element according to claim 1, obtainable by precipitating a coating solution, provided with copper particles and a matrixing agent, onto a substrate, preferably by means of dip coating, to form the contact region.

9. Method for producing a disinfection element according to claim 1, wherein a substrate is coated at least in part with a coating solution provided with the copper particles and a matrixing agent.

10. Method according to claim 9, wherein the coating solution is prepared by stirring the copper particles into a solution of the matrixing agent, and by subsequent homogenisation.

11. Method according to claim 10, wherein the copper particles are stabilised before being introduced into the solution of the matrixing agent.

12. Method according to claim 11, wherein, for the purpose of stabilisation and homogenisation, the copper particles are subjected to a treatment step in which ultrasound is applied to said particles in a mixture a solvent, preferably ethanol, and polyvinylpyrrolidone (PVP).
